(19) United States
(12) Patent Application Publication
(10) Pub. No.: US 2022/0232857 A1
(43) Pub. Date: Jul. 28, 2022

(54) WILDLIFE SUPPLEMENTATION METHODS AND RELATED COMPOSITION

(71) Applicant: Mar-Vo Mineral Company, Inc., Hillsdale, MI (US)

(72) Inventor: David B. Wheeler, Hudson, MI (US)

(21) Appl. No.: 17/582,605

(22) Filed: Jan. 24, 2022

Related U.S. Application Data
(60) Provisional application No. 63/141,728, filed on Jan. 26, 2021.

(51) Int. Cl. A23K 20/22; A61K 45/06; A23K 50/10; A23K 10/30; A23K 20/147; A23K 20/24; A23K 20/26; A23K 20/20; A23K 30/00

(52) U.S. Cl. CPC A23K 20/22; A61K 45/06; A23K 50/10; A23K 10/30; A23K 30/00; A23K 20/24; A23K 20/26; A23K 20/30; A23K 20/147

(57) ABSTRACT

A wildlife supplement and a related method of controlling supplement consumption, reducing mold in a supplement and/or administering a therapeutic agent, with a particular level of sodium, to an animal in the wild at a remote location is provided. The feed supplement can include sodium in an amount of 5% to 80% by weight, and optionally a feed ingredient, attractant, and/or a mineral supplement. The feed supplement can be applied in a remote area, optionally distal from any livestock feeding area. Consumption of the feed supplement by the animal can be self-regulated by the animal via the sodium content. Mold and/or bacterial growth can be limited via the sodium content. The sodium content can entice animals to consume a therapeutic agent in a controlled manner to orally self-vaccinate, treat and/or prevent a disease or condition.

### BACKGROUND OF THE INVENTION

The present invention relates to wildlife supplementation, and more particularly, to the use of salt in feed for promoting the health, vaccination and/or growth of wildlife.

Hunters, naturalists, researchers, conservationists, and others concerned with the health, well-being, and survival of wildlife regularly provide feed and minerals to animals, particularly large game animals, such as ungulates and Cervids, and particularly in challenging climate conditions, where the animals are likely under nutritional or temperature-related stress. Such feed and minerals are placed in the environment of wildlife in solid or liquid form.

Many times, salt is an ingredient in this supplementation activity, and is primarily included to attract the wildlife to a feed or mineral site. Commercially available products for preparing an artificial salt or mineral site have been limited to large 5 to 50 pound blocks or rocks of minerals and bagged mineral powders with a significant salt content of over 80% to 95% in many cases. Typically, the blocks or rocks are placed on the ground and allowed to dissolve by exposure to humidity and precipitation. Bagged mineral powders may be poured directly onto the ground, and optionally mixed into the soil. Wildlife is attracted to the mineral site and consumes the salt by itself in most cases.

The use of salt as an attractant at mineral or feed sites, however, is limited and focuses primarily on using the salt in a mineral product only, and luring the animal to the site. While minerals, such as salt, can fill a void in the animal's diet, and can be used as an attractant, that typically is all the salt is intentionally used for in connection with the wildlife.

Accordingly, there remains room for improvement in the field of wildlife feed and mineral supplementation in connection with sodium.

### SUMMARY OF THE INVENTION

A wildlife supplement and a related method of controlling supplement consumption, reducing mold in a supplement and/or administering a therapeutic agent, with a particular level of sodium, to an animal in the wild at a remote location, are provided.

In one embodiment, a supplement and related method are provided to slow or reduce the consumption of a feed supplement, which can include a feed ingredient and/or a mineral supplement in a remote area, optionally distal from any livestock, agricultural or other domestic feeding area. Consumption of the feed supplement by the animal can be limited or reduced or slowed by including in the feed supplement at least 5% sodium by weight of the feed supplement.

In another embodiment, the amount of sodium in the feed supplement can be at least 10%, at least 15%, at least 20%, at least 25%, at least 30%, at least 35%, at least 40%, at least 50%, about 10%, between 10% and 80%, inclusive, between 10% and 70% inclusive, between 10% and 60% inclusive, between 10% and 50% inclusive, between 10% and 40% inclusive, between 10% and 30% inclusive, between 10% and 25% inclusive, between 10% and 15%, inclusive, between 15% and 60% inclusive, between 15% and 50% inclusive, between 15% and 40% inclusive, between 15% and 30% inclusive, between 15% and 25% inclusive, between 25% and 70% inclusive, between 25% and 60% inclusive, between 25% and 50% inclusive, between 25% and 40% inclusive, between 25% and 30% inclusive, between 9% and 11% inclusive, or between 9% and 10.8% inclusive. With this level of sodium, the wild animal can self-regulate consumption of the feed supplement with which the sodium is admixed due to the level of the sodium mixed with at least one other food ingredient in the feed supplement. Thus, the animal is impaired from overeating the feed supplement due to the salt content in the same.

In still another embodiment, the wild animal can be a mammal from the Cervid family, or other wild ruminant animals. In one example, the mammal can be a Cervid, such as a deer, an elk, a goat, an antelope, or other Cervid. The Cervid can be attracted to the feed supplement for the sodium content, but also can be limited or slowed in consuming the feed supplement, and in particular the other non-mineral, nutritious food contents of the feed supplement, due to the higher sodium content. In many cases, the mammal can supplement its own, self-regulated diet of natural food in the remote area with the feed supplement.

In yet another embodiment, the method can include leaving the feed supplement in the remote area for a period of at least three days so that the wild animal, such as a mammal, finds and/or is attracted to the feed supplement. The feed supplement can include an attractant, such as an aromatic attractant to draw the mammal to the feed site. The attractant can be an aromatic attractant that draws the mammal to the feed site. In some cases, the attractant can draw the animal from more than 50 yards, more than 100 yards, more than 150 yards, or more than 200 yards away from the feed site due to the aroma of the attractant inducing the animal to the feed site.

In even another embodiment, the feed supplement includes between 5% and 50%, inclusive, or about 10%, or between 9% and 11% inclusive, or between 9% and 10.8% inclusive, by weight sodium. The feed supplement can include at least one of a grain ingredient, a protein ingredient and/or a forage ingredient. These ingredients can be admixed with the sodium. As the ingredients are consumed, the sodium is likewise consumed with them. The sodium however, can begin to build in the wild animal's system and cause the mammal to reduce its desire to continue consuming the ingredient even though the animal has not become full due to that ingredient or other components of the feed supplement, such as the grain ingredient, protein ingredient and/or forage ingredient.

In a further embodiment, the feed supplement can be in a rough particulate form, with parts or pieces of the particulate being flowable. In some cases, the method can include dumping the feed supplement from a container, such as a bag, bucket or receptacle, onto a ground surface in the remote area. The feed supplement can flow in particulate form from the container to the ground during such application. In other cases the method can include dumping the feed supplement via an automatic spreader or feeder, so that the feed supplement is dispersed over a larger area and spread out on that larger area.

In still a further embodiment, the method can include leaving the feed supplement in a remote area for at least two weeks before replenishing the feed supplement with additional feed supplement. In some cases, the area can be quite distal from a road or other traversable area, in which case the feed supplement can be left for at least three weeks, at least four weeks, at least five weeks, at least six weeks, at least seven weeks, or at least eight weeks before being replenished. In some cases, the higher sodium content can so limit the consumption of the feed supplement, while still attracting the mammal to the feed site that a portion of the feed supplement still remains at the feed site at the time of replenishment.

The above embodiments of the feed supplement and related method can address overeating of the supplement. By limiting consumption at a per feed site visit basis, the health of the animal can be preserved. In addition, the application of the feed supplement can be cost effective. For example, a large wild herd of Cervids or other wild ruminants can consume large amounts of the supplement too rapidly and make the logistics of transportation to and the cost of maintaining the site prohibitive. If the wildlife mammal or other animals are hungry and have not adjusted their diet to a feed supplement, the animals can have a high likelihood of overeating and causing metabolic issues such as acidosis. This can occur more often with wild Cervids and other wildlife, than with livestock, agricultural or domestic animals, because the wildlife population might be consuming a wide variety of diets completely unknown to the user providing the feed supplement, and the change in diet is the danger. Generally, the feed supplement and related method of the embodiments above can optionally attract and supplement wildlife nutrition, however, the elevated levels of sodium slow the consumption of these products.

In another embodiment, a supplement and related method are provided with a level of sodium that inhibits mold and bacterial growth in the feed supplement for a several week period so that the feed supplement remains edible to a wildlife mammal for at least that period.

In still another embodiment, a method of preserving a wildlife food supplement in a remote area can include placing a feed supplement in a remote area distal from any livestock feeding area; leaving the feed supplement in the remote area for a period of at least two, three or four weeks so that a wild animal is attracted to and consumes the feed supplement at the remote location. The feed supplement can include an amount of sodium as mentioned above, for example, between 5% and 50%, inclusive, by weight sodium, and at least one of a grain ingredient, a protein ingredient and a forage ingredient. The sodium can inhibit mold and bacterial growth in the feed supplement for several weeks.

In yet another embodiment, the feed supplement can include between 10% and 15%, inclusive, by weight sodium. The feed supplement also can include at least 20% by weight of at least one of the grain ingredient, the protein ingredient and the forage ingredient. The wild animal can supplement a diet of natural food in the remote area with the feed supplement.

The above embodiments of the feed supplement and related method can reduce mold and harmful bacterial growth in feed for Cervids, wild ruminants, and other wildlife using elevated salt levels. Where food supplementation includes the use of grains in moist or wet climate conditions, mold, bacteria and fungus growth, all referred to as mold growth herein, can be problematic. Such grain type products can be placed where they are exposed to rain, snow and other moisture, and are often in remote areas where daily or frequent applications of the supplement are not practical. In some cases, mold growth can be a threat to reduce the consumption of the feed supplement as well as to the safety of the wildlife eating the feed supplement. In the above embodiments, the elevated levels of salt can reduce the mold growth, and thus make the feed supplement edible for longer periods and safe for consumption by animals at a remote site, in some cases two, three, four or more weeks beyond the time the supplement would have become inedible, had it not included the elevated salt levels.

In a further embodiment, a supplement and related method are provided with a level of sodium useful to administer a therapeutic agent to an animal in the wild.

In still a further embodiment, the method can include placing a supplement in a remote area distal from any livestock, agricultural or domestic animal feeding area during a period of Spring green-up, the supplement including a therapeutic agent and a predetermined sodium content; leaving the supplement in the remote area for a period of at least three days so that the wild animal is attracted to and consumes the supplement at the remote location due to the sodium content of the supplement. In this manner, the wild animal can self-administer the therapeutic agent in a therapeutically effective amount due based on the predetermined sodium content which can limit the uptake of the therapeutic agent, while optionally encouraging the uptake initially via the attractant nature of the sodium. Optionally, the wild animal is attracted to the supplement to consume it and the therapeutic agent via the sodium, yet the sodium discourages excess intake of the supplement with therapeutic agent. In this way, the sodium can act as an on and off mechanism to get the animal to start consuming and to stop consuming the supplement with therapeutic agent.

In yet a further embodiment, the wild animal can be a mammal, optionally from the Cervid family. In one example, the Cervid can be a deer, an elk, a goat, an antelope, or other Cervid. Optionally, the Cervid can be a whitetail deer. The Cervid can consume the supplement during a Spring green-up, for example, during at least one of the months of March, April, May and June in the Northern Hemisphere.

In another embodiment, the Cervid can crave electrolytes due to a depletion of electrolytes in the Cervid. The Cervid can consume the supplement due to the amount of sodium in it to replenish the electrolytes in the Cervid. In turn, this can cause the Cervid to consume and thus self-administer the therapeutic agent in the supplement.

In even a further embodiment, the therapeutic agent can be a vaccine, an antibiotic and/or a medication. The sodium can be in the form of sodium chloride. The wild animal can be a mammal, for example a wild ruminant, and in particular can be a Cervid that orally consumes the supplement and thus the therapeutic agent.

In another, further embodiment, the therapeutic agent can be a vaccine for Chronic Wasting Disease, Tuberculosis and/or Epizootic Hemorrhagic Disease. The mammal can be a Cervid that orally consumes the supplement and thus the therapeutic agent to address or treat the aforementioned diseases or conditions associated with the same.

The above embodiments of the feed supplement and related method can utilize sodium to control oral application of therapeutic agents, such as vaccines, medicine, binders, antibiotics, soil additives and any other disease prevention materials to Cervids and other wild animals. This can facilitate a high percentage of a target population obtaining an accurate dosage of the therapeutic agent to aide in disease control and prevention. Exemplary diseases, such as Chronic Wasting Disease (CWD), Tuberculous (TB) and Epizootic Hemorrhagic Disease (EHD), and potentially other diseases, can be treated with the supplement herein, via an oral application to wildlife. The supplement can be used to effectively administer an oral application to a wild animal. It sometimes can be helpful to have a high percentage of a particular wild animal population to ingest the product within a predetermined period to improve effectiveness. Additionally, the lower the variability in dosage, amount the more effective and the more cost effective it can be. Oral treatments with the supplement can be achieved even in large populations of the target animals, and can result in the entire population of the target animals to consume the therapeutic agent without over consumption by some of the population. By controlling as much of the sodium intake as possible, the consumption level of sodium can be closely calculated and a very high percentage of the population can consume a very consistent amount of the supplement, and thus the therapeutic agent, based on body weight and season of the year.

These and other objects, advantages, and features of the invention will be more fully understood and appreciated by reference to the description of the current embodiment and the drawings.

Before the embodiments of the invention are explained in detail, it is to be understood that the invention is not limited to the details of operation or to the details of construction and the arrangement of the components set forth in the following description or illustrated in the drawings. The invention may be implemented in various other embodiments and of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the invention to any specific order or number of components. Nor should the use of enumeration be construed as excluding from the scope of the invention any additional steps or components that might be combined with or into the enumerated steps or components.

### DESCRIPTION OF THE CURRENT EMBODIMENTS

The current embodiments are directed to supplements and related methods that can control supplement consumption, reduce mold growth in a supplement and/or administer a therapeutic agent, with a particular level of sodium, to an animal in the wild at a remote location. In one embodiment, the feed supplement can be used to control supplement consumption, and thus prevent overeating of the supplement, which can be detrimental to the health of the animal, and can increase the cost efficiency of the supplementation with a feed. The feed supplement of this embodiment can include sodium in a particular weight percentage of the feed supplement, in addition to a feed ingredient. The sodium can be in the form of sodium chloride, such as common salt. The sodium can be present in the feed supplement in a particular amount. The amount of sodium is deemed to be an effective amount when it functions in its intended role to cause a wild animal to cease eating or intaking the feed supplement after the animal has eaten a certain amount of the feed supplement. This certain amount can be, for example, optionally up to and including 0.25 pounds, up to and including 0.50 pounds, up to and including 0.75 pounds, up to and including 1.00 pounds, up to and including 1.25 pounds, up to and including 1.50 pounds, up to and including 2 pounds, up to and including 3 pounds, up to and including 4 pounds, up to and including 5 pounds or up to and including 7 pounds. That amount can be less than the animal's normal food consumption capacity, which optionally can be 2, 3, 4, 5, 10, or 20 times the amount of feed supplement consumed where the feed supplement has the effective amount of sodium content. The effective amount may vary depending on the animal, and its size. The amount should be sufficient so that the garlic and sulfur leave the animal via sweat for insect repellency. It is believed that where the feed supplement includes between 5% and 50%, inclusive, by weight sodium, and the feed supplement includes at least one of a grain ingredient, for example but not limited to a corn ingredient, a protein ingredient and a forage ingredient, the sodium can prevent overeating of the feed supplement or restrict consumption of the feed supplement.

Exemplary amounts of the sodium by weight of the feed supplement can be optionally at least 5%, at least 10%, at least 15%, at least 20%, at least 25%, at least 30%, at least 35%, at least 40%, at least 50%, between 10% and 80%, inclusive, between 10% and 70% inclusive, between 10% and 60% inclusive, between 10% and 50% inclusive, between 10% and 40% inclusive, between 10% and 30% inclusive, between 10% and 25% inclusive, between 15% and 60% inclusive, between 15% and 50% inclusive, between 15% and 40% inclusive, between 15% and 30% inclusive, between 15% and 25% inclusive, between 25% and 70% inclusive, between 25% and 60% inclusive, between 25% and 50% inclusive, between 25% and 40% inclusive, between 25% and 30% inclusive, between 9% and 11%, inclusive, or between 9% and 10.8%, inclusive. The particular level or amount of sodium can be selected so that the wild animal can self-regulate consumption of the feed supplement due to the level of the sodium mixed with at least one other food ingredient in the feed supplement. Thus, the wild animal is impaired from overeating the feed supplement.

The feed supplement herein can in some cases be distinguished from a simple attractant, a mineral supplement or a straight food source, which can be an amount of edible food with nothing else added, such as alfalfa, corn, grains or the like. The feed supplement herein can include both food ingredient for a wild animal as well as an attractant item for a wild animal. An attractant item may, but does not necessarily, provide dietary nutrition, but otherwise can attract an animal to the feed supplement via an aroma, whereas a food ingredient provides a substantial amount of dietary nutrition. By “substantial” it is meant that major dietary components including at least a carbohydrate source and/or a fat source are included in the feed supplement, whereas other components such as proteins, vitamins, trace minerals, and the like are optionally but not necessarily included. Mineral supplements can concentrate on providing macro minerals such as calcium, phosphorous, sodium, chlorine and potassium, or micro-minerals such as iron, copper, sulfur, zinc, manganese, cobalt, iodine, selenium, molybdenum and chromium. Of course, the feed supplement herein also can be a mineral supplement as well as a feed supplement.

The current embodiments can be in the form of a feed supplement to supplement the natural diet, of natural foods, of a wild animal living in a remote area. The natural foods can include uncultivated plants, foliage, grasses, trees, shrubs, fruits, berries, leaves, orbs or other plants, cultivated or not. The wild animal is not a farm raised or roaming livestock, such as cattle, horses, sheep or goats. Instead, the wild animal can be an animal that lives untended by humans and generally consumes food of its own choosing, without the guidance of or regulation by humans. The remote area where the feed supplement can be placed can be an area that is distal from any livestock feeding area, which can include but is not limited to a fenced pasture, a feed lot, or other human established grazing or other area where livestock or other domesticated or husbandry animals raised or cared for by humans frequent, feed or are generally located. Optionally, the remote area typically is not accessed or used by livestock or other domesticated animals, except perhaps by occasional happenstance. Optionally, the remote area can be in timbered regions, wide open regions, mountainous regions, grasslands, plains, or even among and around farmland and agricultural regions. Further optionally, the remote area can be distal from a fenced pasture, a feed lot or other established grazing or other area where livestock or other domesticated or husbandry animals raised or cared for by humans frequent, feed or are generally located. Even further optionally, the remote area is identified by a user, with reference to a population of Cervids in that remote area. The user can further identify an application site where the feed supplement is to be physically placed within the remote area. That application site can be near Cervid trails, a water source, a bedding area and/or a travel corridor.

The wild animal can be a mammal. The mammal from the Cervid family, formally referred to as the family Cervidae. In one example, the Cervid can be a whitetail deer, a black tail deer, a mule deer, a sika deer, a reindeer, an elk, a moose, a fallow deer, a roe deer, a red deer, a stag, or other Cervid. The Cervid can be attracted to the feed supplement for the sodium content, but also can be limited or slowed in consuming the feed supplement due to the higher sodium content in the effective amount described herein. Consumption of the feed supplement can be orally, by mouth via intake of the feed supplement by the animal.

As mentioned above, the feed supplement can include a feed ingredient, for example, to provide energy. The feed component optionally can be at least one of a grain ingredient, a sugar-containing ingredient, a protein ingredient, a fat ingredient and a forage ingredient. Where used, the grain ingredients can be in the form of whole grain, ground grain, or a grain component. Such a whole grain, ground grain, or a grain component can be a source of carbohydrate, protein, fat, or all of these. One or more whole grains, ground grains, or grain components and combinations thereof can be included in the feed supplement. Grain ingredients and components include bran, germ, endosperm, or portions thereof. Grain ingredients can include natural or genetically engineered grains including but not limited to amaranth, barley, buckwheat, bulgur, corn, millet, oats, rice, rye, sorghum and wheat, such as durum wheat, and bread wheat including hard wheat, soft wheat, white wheat, red wheat, winter wheat, and spring wheat. The grain ingredient can be present in the feed supplement (not including any supplemental, separate amount of the grain ingredient to which the feed supplement is added) at a total amount of optionally about 5% to 75%, inclusive, by weight of the feed supplement, about 10% to 50%, inclusive, by weight of the feed supplement, about 20% to 40%, inclusive, by weight of the feed supplement, about 15% to 30%, inclusive, by weight of the feed supplement, about 10% to about 25%, inclusive, by weight of the feed supplement, about 10% to 25%, inclusive, by weight of the feed supplement, about 15% to 25%, inclusive, by weight of the feed supplement, about 15% to 22%, inclusive, by weight of the feed supplement, about 18% to about 21%, inclusive, by weight of the feed supplement, at least 10% by weight of the feed supplement, at least 15% by weight of the feed supplement, at least 20% by weight of the feed supplement, at least 25% by weight of the feed supplement, at least 50% by weight of the feed supplement or at least 80% by weight of the feed supplement.

Where included in the feed supplement, the sugar-containing ingredient can be a carbohydrate source. Sugar containing ingredients can include, for example, molasses, honey, sugarcane, sugar beets, turnips, fruit, fruit portions, fruit extracts, and the like. Other sugar containing ingredients can be sugars, sugar alcohols, and combinations thereof. Examples of suitable sugars include fructose, glucose, galactose, sucrose, maltose, and lactose, or two or more thereof. Examples of suitable sugar alcohols include propylene glycol, adonitol, allitol, altritol, arabinitol, dulcitol, erythritol, galaxitol, glucitol, glycerol, iditol, inositol, isomalt, lactitol, maltitol, mannitol, perseitol, ribitol, rhamnitol, threitol, and xylitol or two or more thereof. Combinations of various sugar-containing ingredients, sugars, and sugar alcohols can be included in the feed supplement. The sugar containing ingredient can be present in the feed supplement at a total amount of optionally about 1% to 75%, inclusive, by weight of the feed supplement, about 10% to 50%, inclusive, by weight of the feed supplement, about 20% to 40%, inclusive, by weight of the feed supplement, about 15% to 30%, inclusive, by weight of the feed supplement, about 1% to about 12%, inclusive, by weight of the feed supplement, or about 0.1% to about 5%, inclusive by weight of the supplement.

Where included in the feed supplement, the fat ingredient can be in the form of oils, optionally derived from plants. These oils can be expressed oils, distilled oils and/or extracted oils. Some suitable oils can be extracted from the fruits or seeds of plants. Some examples of oils that can be used in the feed supplement can include coconut oil, palm oil, corn oil, flaxseed oil, rice bran oil, peanut oil, rapeseed oil, sesame oil, soybean oil, sunflower oil, wheat germ oil, and mustard oil. The fat ingredient can be present in the feed supplement at a total amount of optionally about 0.1% to 15%, inclusive, by weight of the feed supplement, about 2% to 10%, inclusive, by weight of the feed supplement, about 2% to 5%, inclusive, by weight of the feed supplement, about 1% to 7%, inclusive, by weight of the feed supplement, or about 5% by weight of the feed supplement.

Where included in the feed supplement, the protein ingredient can be from a variety of sources, such as whole or ground legumes and nuts, or an extract, or component thereof. Examples of suitable protein ingredients can include soybeans, peanuts, various common strains of beans and peas, lentils, lima beans, peanuts, walnuts, acorns and the like. The protein ingredient can be present in the feed supplement at a total amount of optionally about 1% to 75%, inclusive, by weight of the feed supplement, about 10% to 50%, inclusive, by weight of the feed supplement, about 20% to 40%, inclusive, by weight of the feed supplement, about 15% to 30%, inclusive, by weight of the feed supplement, or about 18% to about 25%, inclusive by weight of the feed supplement.

Wherein included in the feed supplement, the forage ingredient can be from a variety of sources, such as chopped alfalfa, chopped timothy, beet pulp, alfalfa cubes, alfalfa and timothy cubes, grasses and corn silage and the like. The forage ingredient can be present in the feed supplement at a total amount of optionally about 1% to 75%, inclusive, by weight of the feed supplement, about 10% to 50%, inclusive, by weight of the feed supplement, about 20% to 40%, inclusive by weight of the feed supplement, about 15% to 30%, inclusive by weight of the feed supplement, or about 18% to about 25%, inclusive, by weight of the feed supplement.

In some embodiments, one or more additives can be included in the feed supplement. Such additives optionally can include preservatives, stabilizers, emulsifiers and/or adjuvants that are safe for consumption by deer. The amount of an additive can be selected based on the material and its intended application.

Where included, suitable preservatives can include sorbic acid, potassium sorbate, fumaric acid, propionic acid, acetic acid and benzoic acid. Antimicrobial preservatives can include sorbic acid and its salts, benzoic acid and its salts, calcium propionate, sodium nitrite, sulfites (sulfur dioxide, sodium bisulfite, potassium hydrogen sulfite, etc.) and disodium EDTA. Another group of preservatives can be citric and ascorbic acids in lemon or other citrus juice, as well as Vitamin C and Vitamin E. The preservative can be present in the feed supplement at a total amount of optionally about 0.0001% to 10%, inclusive, by weight of the feed supplement, about 0.001% to 5%, inclusive, by weight of the feed supplement or about 0.001% to 2% inclusive by weight of the feed supplement.

Where included, suitable emulsifiers can include lecithin, mustard seed mucilage, soy lecithin, sodium stearoyl lactylate, and monoglyceride ester of diacetyl tartaric acid. Where included, suitable stabilizers can include tetrasodium pyrophosphate to prevent interaction of calcium ions with other components of the feed supplement. Where included, adjuvants can include attractants such as vitamins, minerals, salts, trace nutrients, or other elements or nutrients beneficial to deer, as well as other palatability improvers, such as Maillard reaction products of various sugars and proteins, as well as natural and artificial flavors. In some embodiments, an attractant in the feed supplement can include an airborne attractant having attractive compounds that are volatile at use temperatures, so the wild animal can detect the airborne compounds from some distance away from the edible composition. In some cases, the attractants can draw the animal from more than 50 yards, more than 100 yards, more than 150 yards, or more than 200 yards away from the feed site due to the aroma of the attractant inducing the animal to the feed site. Examples of suitable attractants include various food odors, such as plant extracts, essential oils of plants known to attract Cervids, nut extracts, seed extracts, volatile esters attributed to the odors of beets, acorns, apples and/or persimmons. The attractant can be present in the feed supplement at a total amount of optionally about 0.0001% to 5% inclusive by weight of the feed supplement, about 0.001% to 2% inclusive by weight of the feed supplement or about 0.001% to 1% inclusive by weight of the feed supplement. In some cases, the sodium content of the supplement can act as the attractant as well.

In some embodiments, one or more sources of fiber can be included in the feed supplement. Such sources of fiber can include wheat or other types of bran, soybean hulls, sugar beet pulp, components of haylage or silage, cottonseeds, canola meal, citrus pulp, hominy, distiller grains, malt sprouts, wheat middlings and sunflower meal to name a few, which may or may not be in combination. The amount of a fiber can be selected based on the material and its intended application. The fiber ingredient can be present in the feed supplement at a total amount of optionally about 1% to 75% inclusive by weight of the feed supplement, about 10% to 50% inclusive by weight of the feed supplement, about 20% to 40% inclusive by weight of the feed supplement, about 15% to 30% inclusive by weight of the feed supplement, about 5% to about 10% inclusive by weight of the feed supplement, or about 7% by weight of the feed supplement.

In some embodiments, one or more minerals and/or vitamins can be included in the feed supplement. Such vitamins and/or minerals can include linoleic acid, vitamin A, vitamin C, vitamin D, vitamin E, vitamin K, thiamine (vitamin B1), riboflavin (vitamin B2), Vitamin B6, vitamin B12, niacin, folic acid, pantothenic acid, calcium (such as calcium iodate, calcium carbonate), magnesium, iron, zinc (such as zinc oxide), manganese, copper, cobalt (such as cobalt carbonate), selenium (such as sodium selenite), phosphorus and iodine. In some cases, biotin, choline and/or inositol can be added. The amount of a vitamin and/or mineral can be selected based on the material and its intended application. Each mineral and/or vitamin can be present in the feed supplement at a total minimum amount of optionally 1 ppm, 2 ppm, 3 ppm, 4 ppm, 5 ppm, 6 ppm, 7 ppm, 8 ppm, 9 ppm, 10 ppm, 11 ppm, 12 ppm, 13 ppm, 14 ppm, 15 ppm, 16 ppm, 17 ppm, 18 ppm, 19 ppm, 20 ppm, 21 ppm, 22 ppm, 23 ppm, 24 ppm, 25 ppm, 26 ppm, 27 ppm, 28 ppm, 29 ppm, 30 ppm, 31 ppm, 32 ppm, 33 ppm, 34 ppm, 35 ppm, 36 ppm, 37 ppm, 38 ppm, 39 ppm, 40 ppm, 41 ppm, 42 ppm, 43 ppm, 44 ppm, 45 ppm, 46 ppm, 47 ppm, 48 ppm, 49 ppm, 50 ppm, 60 ppm, 70 ppm, 80 ppm, 90 ppm, 100 ppm or more, or in increments of 0.1 ppm, 0.2 ppm, 0.3 ppm, 0.4 ppm, 0.5 ppm, 0.6 ppm, 0.7 ppm, 0.8 ppm, 0.9 ppm of any of the foregoing values in the feed supplement, depending on the application.

The following is a first example of the feed supplement of the current embodiment and a list of ingredients: Processed Grain By-Product, Salt, Molasses Products, Calcium Carbonate, Sodium Selenite, Zinc Oxide, Calcium Iodate, Cobalt Carbonate, Natural and Artificial Flavors, Propionic Acid, Acetic Acid, Benzoic Acid, Sorbic Acid and Propylene Glycol. Analysis of the foregoing first example yielded the contents and composition in Table 1 below, with all percentage in percent by weight of the feed supplement.

TABLE 1
Analysis of Feed Supplement Components

| Ingredient | Percent by Weight or PPM |
|---|---|
| Crude Protein | 21.00% |
| Crude Fat | (minimum) 5.00% |
| Crude Fiber | (maximum) 7.00% |
| Calcium (Ca) | (maximum) 2.20% |
| Selenium (Se) | (minimum) 1.19 ppm |
| Calcium (Ca) | (minimum) 1.70% |
| Zinc (Zn) | (minimum) 46.00 ppm |
| Salt (Sodium, NaCl) | 9.00% (minimum) to 10.8% (maximum) |
| Copper (Cu) | (minimum) 4.50 ppm |

The above exemplary feed supplement and its ingredients can be formed using conventional mixing techniques. The ingredients optionally can be in powder, particulate and/or liquid form. When admixed, the ingredients optionally can form a dry, low moisture content powder or particulate. The particulate is generally flowable, which means it can flow out of a container upon dispensation at a particular supplement site, such as a remote feeding site as described above. Typically, the feed supplement is not in a paste, pelletized or liquid form. In a particulate form, the feed supplement can be packaged in a container, such as a bag, a bucket, a tub, a small tote or the like, constructed from polymers, paper or other materials. When in the form of a bag, the container can store different weights of the particulate feed supplement, for example, optionally 10 pounds, 20 pounds, 30 pounds, 40 pounds, 50 pounds, 60 pounds, 70 pounds, 80 pounds, 90 pounds or 90 pounds, depending on the application and the target consumer. Where the container may be carried for long distances, the weight can be less than that weight where the container may be carried a short distance, or dumped from a vehicle, directly on a site. The bag can include an easy open closure, such as a pull strip to facilitate easy opening, and can be lined with a poly bag, and an outer paper cover printed with information about the product.

As mentioned above, the feed supplement can be applied to a remote site known to be frequented by target wild animals, such as Cervids. The feed supplement can be dumped directly onto the ground, flowing out from the bag in particulate form in a pile or in a spread across a large area, 10 foot by 10 foot or some other size, to ensure spacing for multiple animals consuming the feed supplement. Alternatively, the feed supplement can be emptied into a gravity fed or automatic feeder that disperses the feed supplement over an area in a remote location. In some cases, the feed supplement can be placed in front of observation or hunting blinds, stands, or remote cameras in the remote site.

Optionally, the feed supplement can be added to other pure straight feed for the targeted wild animals. For example, the feed supplement can be added directly to a separate quantity of grain ingredients, such as corn, to address deficiencies in the straight feed. For example, the feed supplement can increase protein, add fat, increase calcium and other minerals in the wild animal's feedings on the straight feed. The addition of the feed supplement herein also can reduce the consumption of the straight feed due to the salt content. To demonstrate this, a field trial was conducted at a remote site to compare consumption of the feed supplement, with the ingredients of the first example and analysis content of Table 1 above, to the consumption of straight corn. A pile of about 50 pounds of straight corn was placed at the remote site by itself, and was completely consumed in 24 hours by whitetail deer. During another, different period, a pile of about 20 pounds of the feed supplement of the first example above was placed at the remote site by itself, and was completely consumed in three days by whitetail deer, presumably the same ones that rapidly consumed the straight corn during the other shorter period of one day. Thus, the feed supplement was at least 3 times, or at least 7.5 times (considering the poundage of supplement to straight corn) more efficient at reducing consumption of the feed supplement than straight corn due to its sodium content. In turn, this reduced the number of times a user had to replenish a remote site with the feed supplement versus corn, and reduced the cost of the supplementation program.

Further optionally, the feed supplement can be admixed with a separate quantity of a grain ingredient, sugar containing ingredient, protein ingredient and/or forage ingredient. For example, a user can mix the feed supplement with a quantity of straight corn (a grain ingredient) by admixing the two or simply dumping one on the other, and put the mixture at a remote area as described herein. In some cases, the feed ingredient can include an aromatic attractant. The feed supplement can be mixed in a ratio to the grain ingredient such as corn in a ratio to enhance attraction and consumption. For example, the ratio of the separate quantity of corn, or other grain ingredient, to the feed supplement can be optionally about 20:1, about 15:1, about 10:1, about 9:1, about 8:1, about 7:1, about 6:1, about 5:1, about 4:1, about 3:1, about 2:1, about 1:1, about 20:1 to about 1:1, about 10:1 to about 5:1, or about 9:1.

In another experiment, field trials were conducted at a remote site to determine whether the feed supplement with the ingredients of the first example and analysis content of Table 1 above would reduce the consumption of straight corn when the straight corn was mixed with the feed supplement. During one period, straight corn only was placed at the remote site and consumed by whitetail deer. During another, different period, a mixture of the feed supplement at 20% and straight corn at 80% by weight, was placed at the same remote site, so that the same whitetail deer presumably would consume the mixture. The mixture of feed supplement plus straight corn lasted 2.5 times longer or 3.0 times longer at the remote site than the straight corn of the other period. Thus, the feed supplement limited the consumption of the straight feed it was mixed with, by at least 2.5 times or 3.0 times over the consumption of the straight feed alone. This in turn, limited visits to the remote site to replenish the feed, and the amount of straight corn consumed to realize corresponding cost savings for straight corn supplementation.

The feed supplement of this embodiment can be used in a method that can limit the consumption of the feed supplement via the sodium content present in the feed supplement. It is believed that when the sodium content is elevated enough, to the amounts described herein, the wild animal will naturally become disinterested in consuming more of the feed supplement after consuming only a small amount of it. It is believed that the wild animal self-regulates after intaking the feed supplement with the elevated salt content, perhaps being overwhelmed with the increase in the salt in the animal's gut, or perhaps due to overwhelming the animal's taste with an excessive salty taste.

As an example of limiting food intake, a Cervid, such as a whitetail deer, typically can consume 2 to 4 pounds daily in Spring and Summer in the Northern Hemisphere, and in particular in North America. That amount can increase to 4 to 7 pounds in later Fall and Winter there. At a single feeding at a remote site, deer sometimes can consume 0.5 pounds to 2.5 pounds of straight feed, such as corn or another grain, and perhaps more if it is stressed or has not had access to natural or other food. With the current embodiment, the animal is impaired from overeating the feed supplement. For example, a deer can cease eating or intaking the feed supplement after it has eaten a certain amount of the feed supplement. This certain amount can be, for example, optionally up to and including 0.10 pounds, up to and including 0.25 pounds, up to and including 0.50 pounds, up to and including 0.75 pounds, up to and including 1.00 pounds, up to and including 1.25 pounds, up to and including 1.50 pounds, up to and including 2 pounds, up to and including 3 pounds, up to and including 4 pounds, up to and including 5 pounds or up to and including 7 pounds. It is believed that the amount of sodium, optionally in the form of salt, in the feed supplement can reduce this consumption, yet still facilitate consumption of the feed supplement, can be in the amounts mentioned above, and in some cases, amounts optionally being at least 5%, between 5% and 20% inclusive, between 10% and 20% inclusive, between 10% and 15%, inclusive, or about 10%. Again, these levels can be low enough that the Cervid will still eat the feed supplement and benefit from the other ingredients, but high enough that the Cervid soon becomes disinterested in the feed supplement and moves on to limit consumption at a single feeding at the remote site. Of course, within a period, such as optionally less than 3 hours, less than 6 hours, less than 12 hours, less than 24 hours, less than one day, less than two days, less than three days, less than four days, less than five days, less than six days or less than seven days, the Cervid can return to the remote site to consume the feed supplement again. At that other feeding, the Cervid can again be limited in intake by the sodium content in the feed supplement. Over time, the amount of feed supplement consumed can be curtailed by the salt content. In turn, the feed supplement can keep the animal returning to the site over a longer period of time for multiple feedings because the animal or group of animals does not rapidly consume the entire amount of applied feed supplement. This in turn can reduce the cost of the applications of the feed supplement, and can reduce the number of times that the feed supplement is applied at the remote site, which can reduce effort and travel costs by the user applying the feed supplement.

As mentioned above, the current embodiment of the feed supplement can be used to supplement food consumption of a wild animal, for example, a Cervid in a method. This method can include: placing a feed supplement in a remote area distal from any livestock feeding area; leaving the feed supplement in the remote area for a period of at least three days so that a mammal from the Cervid family is attracted to and consumes the feed supplement at the remote location; limiting the consumption of the feed supplement by the mammal by including in the feed supplement at least 5% sodium by weight of the feed supplement, so that the mammal self-regulates the consumption of the feed supplement due to the level of the sodium mixed with at least one other food ingredient in the feed supplement. With this method, the mammal can be impaired from overeating the feed supplement.

In this method, the feed supplement can include any of the levels of sodium recited above by weight sodium, in addition to the other ingredients mentioned above, such as one or more of a grain ingredient, a corn ingredient, a protein ingredient and a forage ingredient. Where the feed supplement is in a particulate form, the placing step includes dumping the feed supplement from a bag onto a ground surface in the remote area. The feed supplement can be left in the remote area for at least one week, at least two weeks, at least three weeks, at least four weeks, at least five weeks, at least six weeks, at least seven weeks, at least eight weeks or more before replenishing the feed supplement with additional feed supplement. Optionally, via the feed supplement, the mammal can supplement a diet of natural food in the remote area with the feed supplement. The mammal also can self-regulate the amount of the feed supplement consumed at a single feeding due to the intake and reaction to the level of sodium in the feed supplement, which can cause the mammal to stop consuming the feed supplement during the feeding.

In a second embodiment, the feed supplement described above can be implemented in a method to preserve a wildlife food supplement in a remote area. The feed supplement and related method are provided with a level of sodium, as well as other ingredients, as described above. The level of sodium can inhibit mold, fungus and bacterial growth (interchangeably and collectively referred to as mold growth herein) in the feed supplement for a several week or longer period so that the feed supplement remains edible to a wildlife mammal for at least that period.

The method of preserving the feed supplement in a remote area can include placing a feed supplement in a remote area distal from any livestock feeding area; leaving the feed supplement in the remote area for a period of at least two, three, four, five, six, seven, eight, nine, ten, eleven, twelve or more weeks, so that a wild animal is attracted to and consumes the feed supplement at the remote location. The feed supplement can include an amount of sodium as mentioned above, for example, between 5% and 50%, inclusive, or about 10%, by weight sodium, and at least one of a grain ingredient, a fat ingredient, a protein ingredient and a forage ingredient. The sodium can inhibit mold and bacterial growth in the feed supplement for the several weeks. The other ingredients, such as the grain, fat, protein and/or forage ingredients can thereby be preserved by the sodium inhibiting such mold growth.

The sodium used to inhibit the mold growth again can be present in any of the amounts above, and amounts optionally being at least 5%, between 5% and 20% inclusive, between 10% and 20% inclusive, or between 10% and 15%, or about 10% by weight of the composition. Through outdoor testing and application of the feed supplement, it has been observed that mold growth can be reduced in the feed supplement, including the ingredients in Table 1 above, starting at 5% salt levels, and including between 10% and 15% salt levels, inclusive, or about 10% by weight of the feed supplement. These levels apparently can slow the rate of mold growth to allow the consumption of the feed supplement for an additional two to three weeks beyond the time it would have become inedible by the wild animal, such as a whitetail deer, due to the rot, mold or mildew on the feed supplement. In turn, with this preservation, the other feed ingredients, such as the grain, fat, protein and/or forage ingredients can remain palatable and edible for the extended period.

In one example, a portion of the feed supplement, including the ingredients in Table 1 above, was left at an outdoor remote area subject to snow and precipitation in January in the state of Michigan, with no sign of mold or rot detected in or around the feed supplement after four weeks. The portion was barricaded so it could not be consumed.

This embodiment of the feed supplement and related method can reduce mold and harmful bacterial growth in feed for Cervids and other wildlife using elevated salt levels. Where the feed supplement includes grains or forage, and where it is applied at a remote site in moist or wet climate conditions (typical during lawful elk and deer hunting seasons), mold growth can be problematic. The feed supplement can be placed at the remote site and exposed to rain, snow and other moisture, which can promote mold growth. The elevated levels of salt however, can reduce the mold growth, and thus make the feed supplement edible for longer periods and safe for consumption by animals at a remote site, many weeks beyond the time the supplement and other feed ingredients therein would have become inedible.

The feed supplement in this embodiment can be enhanced with the elevated amounts of sodium or salt as mentioned above to prevent the growth of particular molds, in particular which can grow in the feed supplement given the grain and forage ingredients mentioned above. The application of the sodium levels in the feed supplement can prevent the growth of Fusarium, Aspergillus, and Penicillin molds over a period of time as mentioned above in the feed supplement. In turn, this can reduce the risk that one or more of these molds produce their associated toxins, which can harm or kill the wild animal consuming the feed supplement. As an example, the elevated sodium content can inhibit, impair and/or prevent the growth of Aspergillus, and thus its production of aflatoxins, which is dangerous to the health of the wild animal if eaten.

In another embodiment, the feed supplement described above and a related method can be provided with a level of sodium useful to administer a therapeutic agent to an animal in the wild. Optionally, the feed supplement can include sodium (and in some cases, minerals) and the therapeutic agent, but not the other feed ingredients, such as grain, fat, protein ingredients, or others as mentioned above, despite being called a “feed” supplement. The therapeutic agent can be added to the feed supplement in a dosage appropriate to administer to the wild animal, such as the Cervids described above, or other animals. The therapeutic agent can be a vaccine for Chronic Wasting Disease, Prion Diseases, Tuberculosis, Blue Tongue, and/or Epizootic Hemorrhagic Disease. Some exemplary vaccines can include inactivated poliovirus vaccine, oral poliovirus vaccine, PNEU-VAC-2 and ECO-VAC, commercially available from Cervid Solutions, LLC of Pennsylvania, USA, TB vaccine (V7) containing heat-killed Mycobacterium vaccae (NCTC 11659), other oral TB vaccination agents, oral anthrax vaccination agents, Ebola vaccination agents, porcine epidemic diarrhea virus vaccination agents, H9N2 subtype low pathogenicity avian influenza virus vaccination agents, chronic wasting disease vaccination agents, blue tongue vaccination agents, and/or epizootic hemorrhagic disease agents. Such therapeutic agents can be orally administered to an animal in the supplement herein, with the sodium, to provide a therapeutic effect and/or vaccination with regard to a given virus. Alternatively or in addition, the therapeutic agent can be an antibiotic. Some suitable antibiotics include Terramycin, Enroflaxin, Oxytetracycline, Tetracycline, Trimethoprim sulfa, Penicillin, Amoxicillin and other antibiotics.

Optionally, the feed supplement with the therapeutic agent can be formulated as described herein, but can include an oral rabies vaccine. That feed supplement can be used to control the spread of rabies. The wild animal that can be targeted can be coyotes, wolves, raccoons, feral pigs, rodents, feral cats, wild dogs and other mammals. The rabies vaccine can be orally administered using the methods herein.

These therapeutic agents, whether a vaccine, antibiotic or other medicament, can be included in amounts in the feed supplement suitable to reproduce appropriate therapeutic amounts or dosages per unit body weight for the Cervid or other wild animal orally intaking the feed supplement. For example, the therapeutic agent can be present in the feed supplement, and the sodium content predetermined or calibrated to limit or encourage consumption of the feed supplement, so as to provide a therapeutically effective amount of the therapeutic agent. A therapeutically effective amount means an amount that impairs, suppresses, stops or prevents symptoms and/or progression of infections, diseases and harmful conditions in an animal.

Optionally, the predetermined sodium content can promote consumption of the supplement, and thus an oral dosage of the therapeutic agent per feeding or consumption of the supplement of optionally 5 mg/kg body weight, 10 mg/kg body weight, 15 mg/kg body weight, 20 mg/kg body weight, 25 mg/kg body weight, 30 mg/kg body weight, 40 mg/kg body weight, 50 mg/kg body weight, 55 mg/kg body weight, 60 mg/kg body weight, 70 mg/kg body weight, 75 mg/kg body weight, 80 mg/kg body weight, 85 mg/kg body weight, 90 mg/kg body weight, 95 mg/kg body weight, 100 mg/kg body weight, 150 mg/kg body weight, 200 mg/kg body weight, 250 mg/kg body weight, 300 mg/kg body weight, 350 mg/kg body weight or 400 mg/kg body weight, or other dosages depending on the agent and the animal. The dosages and administration of the therapeutic agent, however, is unmetered and uncontrolled by human intervention, that is, a human does not know how much of the therapeutic agent or how many doses a particular animal takes, and therefore cannot really control the administration to the animal other than by placing or removing the feed supplement from the remote site.

In this embodiment, the method can generally include placing a feed supplement in a remote area distal from any livestock feeding area during a period of Spring green-up, the supplement including a therapeutic agent and a predetermined sodium content, and optionally a feed ingredient; and leaving the supplement in the remote area for a period of at least three days so that the wild animal is attracted to and consumes the supplement at the remote location due to the sodium content of the supplement. In this manner, the wild animal can self-administer the therapeutic agent in a therapeutically effective amount due based on the predetermined sodium content.

As mentioned above, the wild animal optionally can be of the Cervid family, and can be any of those described herein. The supplement can be strategically used and placed in the remote site during a Spring green-up, for example, during at least one of the months of March, April, May and June in the Northern Hemisphere to maximize the potential that the therapeutic agent will be consumed and thus orally self-administered by the animals using the remote site. During Spring green-up, a Cervid typically craves electrolytes, for example, sodium, due to a depletion of electrolytes in the Cervid. The Cervid can consume the supplement due to the amount of sodium in it to replenish the electrolytes in the Cervid. In turn, this can cause the Cervid to consume and thus self-administer the therapeutic agent in the supplement.

Optionally, the method of administering the supplement and its therapeutic agent can be planned before Spring green up. For example, the feed supplement of any of the embodiments herein, but without the therapeutic agent, can be placed in fall, winter, or late winter, to establish a remote site where the animals will return time and time again to consume the feed supplement. After the remote site is established, the feed supplement including the therapeutic agent can be placed at the remote site in early spring with the appropriate dosage of the therapeutic agent in the supplement per pound of salt. This can ensure suitable accuracy of administration and population coverage as soon as the wild animals, such as Cervids, begin to visit the site during Spring green up.

The average sodium or salt intake for an average size whitetail deer, for example, is 5.5 pounds for an entire year. The consumption of sodium or salt will increase approximately six-fold between pre-green up in late winter and two weeks after grass and other forage starts growing aggressively during what is referred to as Spring green up. This is because of the moisture level of the Cervid's diet. Their diet changes from dry grass and browse, about 50% dry matter, to all new grass and forage. The new grass and forage that they begin to eat is almost 90% water, making only 10% dry matter diet. This essentially flushes the electrolytes out of the animal's system. As a result, the Cervid craves the sodium to replenish their level of electrolytes. Accordingly, this period is when administration of the therapeutic agent with the feed supplement of the current embodiments is favorable. During this period, the highest percentage of a population will naturally consume a predictable amount of salt, making it an efficient modality to administer any therapeutic agent orally to wild animals, such as Cervids.

The following additional Statements illustrate further embodiments, the numbering of which is not to be construed as designating levels of importance. Moreover, it is to be understood that the Statements of embodiments recited below are provided in conjunction with and in addition to the embodiments described above, as well as those claimed even farther below. Alternative embodiments that result from combining, integrating, and/or omitting features of the embodiments of the Statements below or any other embodiment described herein may also be within the scope of the present invention.

Statement A: A method of controllably supplementing food consumption of mammals, the method comprising: providing a feed supplement in a remote area distal from any livestock feeding area; leaving the feed supplement in the remote area for a period so that a mammal is attracted to and consumes the feed supplement at the remote location; limiting the consumption of the feed supplement by the mammal by including in the feed supplement a quantity of sodium in the feed supplement.

Statement B: The method of Statement A, wherein the mammal self regulates the consumption of the feed supplement due to the level of the sodium mixed with at least one other food ingredient in the feed supplement, wherein the sodium both attracts the mammal to start consumption of the food ingredient and encourages the mammal to stop consumption of the food ingredient so the mammal is impaired from overeating the feed supplement.

Statement C: The method of any preceding Statement, wherein the feed supplement includes sodium in an amount between 5% and 11%, inclusive, by weight of the feed supplement.

Statement D: The method of any preceding Statement, wherein the feed supplement includes at least one of a grain ingredient, a sugar containing ingredient, a protein ingredient and a forage ingredient.

Statement E: The method of any preceding Statement, wherein the feed supplement includes a grain ingredient including corn in the form of shelled kernels of corn or cracked corn.

Statement F: The method of any preceding Statement, wherein the feed supplement includes a grain ingredient in an amount of about 10% to about 75% by weight of the feed supplement and a protein ingredient in an amount of about 5% to about 25% by weight of the feed supplement.

Statement G: The method of any preceding Statement, comprising adding or admixing the feed supplement with a separate quantity of at least one of a grain ingredient, a sugar containing ingredient, a protein ingredient and a forage ingredient, wherein the feed ingredient enhances the attraction of the mammal to the separate quantity.

Statement H: The method of any preceding Statement, wherein the separate quantity includes corn kernels or cracked corn, wherein the ratio of corn to feed supplement is between 1:1 and 50:1, and optionally about 9:1.

Statement I: The method of any preceding Statement, wherein the feed supplement includes at least one of a grain ingredient, a sugar containing ingredient, a protein ingredient and a forage ingredient, wherein the protein ingredient is at least 20% by weight of the feed supplement, wherein the grain ingredient is at least 40% by weight of the feed supplement.

Statement J: The method of any preceding Statement, wherein the feed supplement is placed by dumping the feed supplement from a bag, or spreading the feed supplement via an automatic feeder, or containing the feed supplement in a container that is accessible to the mammal.

Statement K: The method of any preceding Statement, comprising identifying the remote area including a population of Cervids and locating an application site in the remote area before said providing.

Statement L: A method of administering a therapeutic agent to a wild animal, the method comprising: placing a supplement in a remote area distal from any livestock feeding area, the supplement including a therapeutic agent and a predetermined sodium content; leaving the supplement in the remote area for a period of at least three days so that a wild animal is attracted to and consumes the supplement at the remote location due to the predetermined sodium content of the supplement; wherein the wild animal self-administers the therapeutic agent in a therapeutically effective amount due based on the predetermined sodium content.

Statement M: The method of Statement L, wherein the mammal is a wild ranging ruminant, optionally a Cervid.

Statement N: The method of any preceding Statement, wherein the predetermined sodium content is at least 5% to 15% by weight of the supplement.

Statement O: The method of any preceding Statement, wherein the predetermined sodium content attracts the wild animal so that the wild animal begins consumption of the supplement to ingest orally the therapeutic agent, wherein the consumption of a portion of the predetermined sodium content causes the wild animal to cease consumption of the supplement.

Statement P: The method of any preceding Statement, wherein the placing occurs during Spring green-up in the Northern Hemisphere.

Statement Q: The method of any preceding Statement, wherein the wild animal is at least one of a raccoon, a coyote, a wolf, a feral cat and a wild dog, wherein the wild animal consumes the supplement adjacent a ground surface in the remote area.

Statement R: The method of any preceding Statement, wherein the therapeutic agent is at least one of a vaccine, an antibiotic and a medication.

Statement S: The method of any preceding Statement, wherein the wild animal is a raccoon, wherein the therapeutic agent is an oral rabies vaccine, wherein the raccoon orally consumes the supplement with at least one grain ingredient included in the supplement.

The terms “comprising” or “comprise” are used herein in their broadest sense to mean and encompass the notions of “including,” “include,” “consist(ing) essentially of,” and “consist(ing) of. The use of “for example,” “e.g.,” “such as,” and “including” to list illustrative examples does not limit to only the listed examples. Thus, “for example” or “such as” means “for example, but not limited to” or “such as, but not limited to” and encompasses other similar or equivalent examples. The term “about” as used herein serves to reasonably encompass or describe minor variations in numerical values measured by instrumental analysis or as a result of sample handling. Such minor variations may be in the order of ±0-10, ±0-5, or ±0-2.5, % of the numerical values. Further, the term “about” applies to both numerical values when associated with a range of values. Moreover, the term “about” may apply to numerical values even when not explicitly stated.

Generally, as used herein a hyphen “-” or dash “-” in a range of values is “to” or “through”; a “>” is “above” or “greater-than”; a “≥” is “at least” or “greater-than or equal to”; a “<” is “below” or “less-than”; and a “≤” is “at most” or “less-than or equal to.” On an individual basis, each of the aforementioned applications for patent, patents, and/or patent application publications, is expressly incorporated herein by reference in its entirety in one or more non-limiting embodiments.

Any ranges and subranges relied upon in describing various embodiments independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

The above description is that of current embodiments of the invention. Various alterations and changes can be made without departing from the broader aspects of the invention as defined in the appended claims, which are to be interpreted in accordance with the principles of patent law including the doctrine of equivalents. This disclosure is presented for illustrative purposes and should not be interpreted as an exhaustive description of all embodiments of the invention or to limit the scope of the claims to the specific elements illustrated or described in connection with these embodiments. For example, and without limitation, any individual element(s) of the described invention may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Further, the disclosed embodiments include a plurality of features that are described in concert and that might cooperatively provide a collection of benefits. The present invention is not limited to only those embodiments that include all of these features or that provide all of the stated benefits, except to the extent otherwise expressly set forth in the issued claims. Any reference to claim elements in the singular, for example, using the articles “a,” “an,” “the” or “said,” is not to be construed as limiting the element to the singular. Any reference to claim elements as “at least one of X, Y and Z” is meant to include any one of X, Y or Z individually, any combination of X, Y and Z, for example, X, Y, Z; X, Y; X, Z; Y, Z, and/or any other possible combination together or alone of those elements, noting that the same is open ended and can include other elements.

What is claimed is:

1. A method of controllably supplementing food consumption of Cervids, the method comprising:
placing a feed supplement in a remote area distal from any livestock feeding area;
leaving the feed supplement in the remote area for a period of at least three days so that a mammal from the Cervid family is attracted to and consumes the feed supplement at the remote location;
limiting the consumption of the feed supplement by the mammal by including in the feed supplement at least 5% sodium by weight of the feed supplement, so that the mammal self regulates the consumption of the feed supplement due to the level of the sodium mixed with at least one other food ingredient in the feed supplement,
whereby the mammal is impaired from overeating the feed supplement.

2. The method of claim 1,
wherein the feed supplement includes sodium in an amount between 5% and 50%, inclusive, by weight of the feed supplement,
wherein the feed supplement includes at least one of a grain ingredient, a sugar containing ingredient, a protein ingredient and a forage ingredient.

3. The method of claim 2,
wherein the feed supplement is in a particulate form,
wherein the placing step includes dumping the feed supplement from a container onto a ground surface in the remote area.

4. The method of claim 3,
wherein the sodium is in the form of sodium chloride,
wherein the feed supplement includes at least one of a grain ingredient, a sugar containing ingredient, a protein ingredient and a forage ingredient in an amount that is at least 20% by weight of the feed supplement.

5. The method of claim 4,
wherein the feed supplement includes calcium, phosphorus, magnesium and selenium.

6. The method of claim 5, comprising:
leaving the feed supplement in the remote area for at least two weeks before replenishing the feed supplement with additional feed supplement.

7. The method of claim 2,
wherein the feed supplement includes sodium in an amount between 9% and 10.8%, inclusive, by weight of the feed supplement.

8. The method of claim 7,
wherein the feed supplement includes at least one of a grain ingredient, a sugar containing ingredient, a protein ingredient and a forage ingredient, in an amount of at least 15% by weight of the feed supplement,
wherein the mammal supplements a diet of natural food around the remote area with the feed supplement.

9. A method of administering a therapeutic agent to a Cervid, the method comprising:
placing a supplement in a remote area distal from any livestock feeding area during a period of Spring green-up, the supplement including a therapeutic agent and a predetermined sodium content;
leaving the supplement in the remote area for a period of at least three days so that a Cervid is attracted to and consumes the supplement at the remote location due to the predetermined sodium content of the supplement attracting the Cervid;
wherein the Cervid self-administers the therapeutic agent in a therapeutically effective amount due to the predetermined sodium content limiting the Cervid's consumption of the supplement.

10. The method of claim 9,
wherein the Cervid is a whitetail deer,
wherein the Cervid consumes the supplement adjacent a ground surface in the remote area,
wherein the supplement is in the remote area during the period of Spring green-up which is during at least one of the months of March, April, May and June in the Northern Hemisphere.

11. The method of claim 9,
wherein the therapeutic agent is at least one of a vaccine, an antibiotic and a medication.

12. The method of claim 11,
wherein the predetermined sodium content is in the form of sodium chloride present in the supplement.

13. The method of claim 12,
wherein the Cervid is a whitetail deer,
wherein the Cervid orally consumes the supplement.

14. The method of claim 12,
wherein the Cervid is an elk,
wherein the Cervid orally consumes the supplement.

15. The method of claim 9,
wherein the therapeutic agent is a vaccine for at least one of Chronic Wasting Disease, Tuberculosis and Epizootic Hemorrhagic Disease.

16. The method of claim 9,
wherein the Cervid craves electrolytes due to a depletion of electrolytes in the Cervid during the period of the Spring green-up,
wherein the Cervid consumes the supplement due to the predetermined amount of sodium to replenish the electrolytes in the Cervid, which thereby causes the Cervid to also consume the therapeutic agent in the supplement.

17. The method of claim 16,
wherein the therapeutic agent is at least one of a vaccine, an antibiotic and a medication,
wherein the Cervid consumes the supplement, which is in particulate form when placed from a ground surface.

18. A method of preserving a wildlife feed supplement in a remote area, the method comprising:
placing a feed supplement in a remote area distal from any livestock feeding area;
leaving the feed supplement in the remote area for a period of at least four weeks so that a wildlife mammal is attracted to and consumes the feed supplement at the remote location, the feed supplement comprising sodium in an amount of between 5% and 50%, inclusive, by weight of the feed supplement, and at least one of a grain ingredient, a sugar containing ingredient, a protein ingredient and a forage ingredient,
wherein the sodium inhibits mold and bacterial growth in the feed supplement for at least four weeks so that the feed supplement remains edible to the wildlife mammal for at least the four weeks.

19. The method of claim 18,
wherein the wildlife mammal is a Cervid,
wherein the Cervid consumes the supplement in particulate form from a ground surface.

20. The method of claim 19,
wherein the sodium is in an amount between 10% and 25%, inclusive, by weight of the feed supplement,
wherein the at least one of the grain ingredient, the sugar containing ingredient, the protein ingredient and the forage ingredient is in an amount that is at least 15% by weight of the feed supplement,
wherein the wildlife mammal supplements a diet of natural food in the remote area with the feed supplement.
